(19) United States
(12) Patent Application Publication
(10) Pub. No.: US 2024/0252518 A1
(43) Pub. Date: Aug. 1, 2024

(54) Cannabinoid Formulations and Methods of Use

(71) Applicant: Jerome Irving, Glenn Mills, PA (US)

(72) Inventor: Jerome Irving, Glenn Mills, PA (US)

(21) Appl. No.: 18/505,092

(22) Filed: Nov. 8, 2023

Related U.S. Application Data
(60) Provisional application No. 63/382,813, filed on Nov. 8, 2022.

(51) Int. Cl. A61K 31/00; A61K 9/00; A61K 47/14; A61K 47/44

(52) U.S. Cl. CPC A61K 31/658; A61K 9/0056; A61K 47/14; A61K 47/44

(57) ABSTRACT

Disclosed herein is at least one cannabinoid formulation and at least one method of using a formulation disclosed herein including at least one pharmaceutical use. Further disclosed herein are methods of making at least one cannabinoid disclosed herein.

### BRIEF SUMMARY

Provided herein is an edible oil comprising:
1) from about 32 mg/ml to about 50 mg/ml of cannabigerol (CBG);
2) from >about 32 mg/ml of cannabidiol (CBD); and
3) from about 20 mg/ml to about 50 mg/ml of cannabidivarin (CBDV).

Further provided herein is a composition as recited above further comprising from about 32 mg/ml to about 50 mg/ml of THC (Δ⁸) acetate ester (“THC-O-acetate”).

Further provided herein is a method of making a cannabinoid composition comprising
1) extracting:
   (a) CBG from a cannabis Flower enriched in CBG to attain a crude oil CBG extract comprising from about 55% up to about 88% by weight of the total weight of the oil;
   (b) CBD from a cannabis Flower enriched in CBD to attain a crude oil CBD extract comprising from about 55% up to about 88% by weight of the total weight of the oil; and
   (c) CBDV from a cannabis Flower enriched in CBDV to attain a crude oil CBDV extract comprising from about 55% up to about 88% by weight of the total weight of the oil;
2) distilling:
   (a) the CBG extract to attain a CBG distillate comprising from about 75% up to about 92% CBG by weight of the total weight of the distillate.
   (b) the CBD extract to attain a CBD distillate comprising from about 75% up to about 92% CBD by weight of the total weight of the distillate.
   (c) the CBDV extract to attain a CBDV distillate comprising from about 75% up to about 92% CBDV by weight of the total weight of the distillate; and
3) mixing the CBG distillate, the CBD distillate, and the CBDV distillate into an edible oil to form a composition comprising about 32 mg/ml to about 50 mg/ml of CBG, about >32 mg/ml CBD, and about 20 mg/ml to about 50 mg/ml of CBDV.

Further provided herein is a method as recited above further comprising mixing THC-O-acetate into the edible oil to a attain a composition comprising about 32 mg/ml to about 50 mg/ml of CBG, about >32 mg/ml CBD, about 20 mg/ml to about 50 mg/ml of CBDV and about 32 mg/ml to about 50 mg/ml THC-O-acetate.

In another aspect, CBG distillate, CBD distillate, and CBDV distillate are mixed into an edible oil to form a composition comprising about 32 mg/ml to about 50 mg/ml each of CBG, CBD, CBDV.

Further provided herein is a method as recited above further comprising mixing THC-O-acetate into an edible oil as recited above to a attain a composition comprising about 32 mg/ml to about 50 mg/ml of CBG, CBD, CBDV and THC-O-acetate.

Further provided herein is a product made by the processes recited above.

Further provided herein is a method of reducing joint, muscle and/or tendon soreness associated with exercise or trauma, in a Subject suffering from such soreness, by administering to the Subject up to about at least 1 ml up to about 2 ml daily, of a composition provided herein. The composition is particularly provided as a unit dose of 1 ml.

Further provided herein is a method of reducing insomnia and/or sleep disruption in a Subject by administering to the Subject in need of reduced insomnia and/or sleep disruption up to about at least about 1 ml, particularly nightly, of a composition provided herein. The composition is particularly provided as a unit dose of 1 ml.

Further provided herein is a method of increasing sexual arousal in a male Subject by administering to the Subject in need of such increased arousal up to about at least 1 ml, particularly daily, of a composition as provided herein The composition is particularly provided as a unit dose of 1 ml.

Further provided herein is a method of inducing weight loss in a Subject by administering to the Subject in need of such weight loss up to about at least 1 ml, particularly daily, of a composition as recited herein. The composition is particularly provided as a unit dose of 1 ml.

Also provided herein is a method of alleviating pain and/or abdominal discomfort in a Subject who experiences indigestion, gas, IBS, diverticulitis and/or GERD by administering up to about at least 1 ml, particularly daily, of a composition as provided herein to the Subject in need thereof. The composition is particularly provided as a unit dose of 1 ml.

### DETAILED DESCRIPTION

In one embodiment a composition described herein comprises CBG in an amount of from about 33 mg/ml to about 47 mg/ml, more particularly from about 36 mg/ml to about 45 mg/ml, and more particularly in an amount of about 42 mg/ml. In one aspect, the CBG is provided in an amount of about 39 mg/ml.

In one embodiment a composition provided herein comprises CBD in an amount of from about 33 mg/ml to about 96 mg/ml, more particularly from about 36 mg/ml to about 96 mg/ml, and more particularly in an amount of about 96 mg/ml.

In one embodiment a composition provided herein comprises CBD in an amount of from about 33 mg/ml to about 47 mg/ml, more particularly from about 36 mg/ml to about 45 mg/ml, and more particularly in an amount of about 42 mg/ml.

In yet another embodiment a composition provided herein comprises CBDV in an amount of from about 20 mg/ml to about 47 mg/ml, more particularly from about 36 mg/ml to about 45 mg/ml, and more particularly in an amount of about 22 mg/ml.

In yet another embodiment a composition provided herein comprises CBDV in an amount of from about 33 mg/ml to about 47 mg/ml, more particularly from about 36 mg/ml to about 45 mg/ml, and more particularly in an amount of about 42 mg/ml.

In still yet another embodiment a composition provided herein comprises THC-O-acetate in an amount from about 33 mg/ml to about 47 mg/ml, more particularly from about 36 mg/ml to about 45 mg/ml, and more particularly in an amount of about 42 mg/ml. In one aspect, the THC-O-acetate is provided in an amount of about 36 mg/ml.

In still another embodiment a composition provided herein comprises CBDA in an amount of <0.7%, more particularly from 0.7% up to about 2.0% of the total weight of the composition. In another embodiment the concentration of CBDA in a composition provided herein is about 7 mg/ml up to about 20 mg/ml, more particularly about 16 mg/ml.

In another embodiment the edible oil is a vegetable oil, plant oil or animal fat. More particularly the edible oil is a medium chain triglyceride (MCT). In a particular embodiment provided herein the MCT is derived from coconut oil.

In another embodiment the edible oil comprises hemp oil. In a particular embodiment the hemp oil is derived from a hemp Flower. In a further embodiment, the hemp oil is derived from a hemp seed.

In one aspect CBG, CBD and/or CBDV is extracted from a cannabis Flower enriched in CBG, CBD and/or CBD to attain a crude oil comprising from about 55% to about 88% (w/w) of CBG, CBD and/or CBDV.

In yet another embodiment, the CBG distillate comprises from about 75% up to about 92% CBG by weight of the total weight of the distillate, more particularly from about 85% up to about 91%.

In yet another embodiment, the CBD distillate comprises from about 75% up to about 92% CBD by weight of the total weight of the distillate, more particularly from about 85% up to about 91%.

In yet another embodiment, the CBDV distillate comprises from about 75% up to about 92% CBDV by weight of the total weight of the distillate, more particularly from about 85% up to about 91%.

In a further embodiment, the methods set forth herein comprise mixing into an edible oil CBG, CBD, and CBDV in an amount to attain a concentration of about 32 mg/ml to about 50 mg/ml CBG, about > or equal to 32 mg/ml CBD, and about 20 mg/ml to about 50 mg/ml CBDV. In another aspect, the CBG is provided in a an amount of about 36 mg/ml to about 45 mg/ml, more particularly in an amount of about 42 mg/ml. In another aspect, the CBG is provided in an amount of about 39 mg/ml. In a further embodiment, the CBDV is provided in an amount of about 22 mg/ml.

In another aspect, the CBD is provided in an amount of about 32 mg/ml to about 96 mg/ml, more particularly in an amount of about 96 mg/m. In another aspect. THC-O-acetate is added to the edible oil comprising CBG, CBD, and CBDV to attain a concentration of about 32 mg/ml to about 50 mg/ml, particularly from about 36 mg/ml to about 45 mg/ml, more particularly about 42 mg/ml THC-O-acetate.

In a further embodiment, the methods set forth herein comprise mixing into an edible oil CBG, CBD, and CBDV in an amount to attain a concentration of about 32 mg/ml to about 50 mg/ml, particularly from about 36 mg/ml to about 45 mg/ml, more particularly about 42 mg/ml of each cannabinoid. In another aspect, the CBG is added to the to the oil to attain a concentration of about 39 mg/ml. In a further aspect the CBD is provided in an amount of about 95 mg/ml. In a further embodiment, the CBDV, is provided in an amount of about 22 mg/ml.

In another embodiment the THC-O-acetate is added to the edible oil comprising CBG, CBD, and CBDV to attain a concentration of about 32 mg/ml to about 50 mg/ml, particularly from about 36 mg/ml to about 45 mg/ml, more particularly about 42 mg/ml THC-O-acetate. In another aspect the THC-O is added to the edible oil to attain a concentration of about 36 mg/ml.

In one aspect, the cannabinoids described herein are extracted from cannabis Flower by suitable solvents known in the art. In a further aspect the solvents used for extraction may include but are not limited to ethanol, propane, butane, pentane, heptane and CO₂. In a particular embodiment the solvent comprises ethanol. In one particular embodiment the solvent is chilled ethanol particularly chilled to about −20° C. or colder.

In one embodiment, the Flower comprising the cannabinoids described herein is extracted via a centrifuge extractor wherein the Flower is placed into a mesh cage placed into chilled ethanol. The extract, for each cannabinoid, is collected and further distilled to form an oil with a concentrated cannabinoid suitable to be added to an edible oil to produce the compositions described herein.

Flower as used herein is a flower of a cannabis plant, with or without seeds.

The expression “Flower enriched” refers to a Flower generated from a plant that contains a higher amount of a particular cannabinoid through plant varieties that are hybrids or genetically manipulated to produce a higher amount of the cannabinoid. In a particular embodiment, the cannabinoids are extracted from Flower enriched in the particular cannabinoid sought. In a more particular embodiment, the enriched flower comprises from about 10% up to about 39% of CBG, CBD and/or CBDV by weight of the total weight of the flower.

The term “Subject” as used herein refers to a human subject.

In one aspect, the distillate enriched in CBD comprises less than 10%, further particularly less than 5%, more particularly less than 0.5%, even more particularly less than 0.1% total cannabinoid (other than CBD).

In one aspect, the distillate enriched in CBG comprises less than 10%, further particularly less than 5%, more particularly less than 0.5%, even more particularly less than 0.1% total cannabinoids (other than CBG).

In another aspect, the distillate enriched in CBDV comprises less than 10%, further particularly less than 5%, more particularly less than 0.5%, even more particularly less than 0.1% total cannabinoids (other than CBDV).

In another aspect a commercially available oil enriched in THC-O-acetate comprises less than 0.5% total cannabinoids, more particularly less than 0.1% total cannabinoids and even more particularly less than 0.01% total cannabinoid (other than THC-O-acetate).

In yet another aspect a composition is provided comprising CBD, CBG and CBDV and less than 5%, more particularly less than 1%, even more particularly less than 0.1%, yet even more particularly less than 0.01% total cannabinoids (other than CBD, CBG and CBDV).

In yet a further another aspect a composition is provided comprising CBD, CBG, CBDV and THC-O-acetate and less than 5%, more particularly less than 1%, even more particularly less than 0.1%, yet even more particularly less than 0.01% total cannabinoids (other than CBD, CBG, CBDV and THC-O-acetate).

The compositions provided herein can also be made from pure (>98%, more particularly >99%) cannabinoid isolates of CBD, CBDV, CBG and THC-O-acetate.

The Examples provided below are for illustrative purposes only and are not meant to limit the scope of any invention described in this application.

### EXAMPLES

### Example 1

Add 5,000 lbs. of cannabis Flower having a CBD content of at least 9% (w/w) of the total cannabinoid weight in the flower. The flower is added to a mesh cage and is placed into an industrial sized centrifuge extractor having about 40 gallons of ethanol chilled to about to at least −20° C. or colder. The extraction process is run for about 6 hours to obtain about 6 liters (captured via gravity into a suitable vessel) of a crude oil comprising about 55 to about 88% (w/w) CBD. The crude oil is heated via indirect heating (e.g., a double boiler) for 6 minutes at a temperature from about 112° C. to 120° C. to lower the viscosity to provide a free-flowing oil. The crude oil is then subject to fractional distillation to obtain a distillate having a CBD content ranging from about 75% to about 92%.

The process is repeated for cannabis Flower enriched in CBG to attain a crude oil extract of from about 55% to about 88% (w/w) CBG and a distillate comprising a CBG content ranging from 75% to about 92%.

The process is repeated for cannabis Flower enriched in CBDV to attain a crude oil extract of from about 55% to about 88% CBDV and a distillate comprising a CBDV content ranging from about 75% to about 92%.

The three distillates are mixed into an edible MCT oil (obtained from coconut oil) to obtain a formulation (tincture) having about 42 mg/ml each of CBG, CBD and CBDV.

### Example 2

The distillates, as made in Example 1, are added, along with a THC-O-acetate distillate, into an edible MCT oil (from coconut oil) to obtain a formulation having about 42 mg/ml each of CBD, CBG, CBDV and THC-O-acetate.

### Example 3

The three distillates, as made in Example 1, are added to a hemp seed oil to attain a concentration of about 39 mg/ml CBG, about 95 mg/ml of CBD and about 22 mg/ml of CBDV.

### Example 4

THC-O acetate is added to the oil of Example 3 to give a THC-O concentration of about 36 mg/ml.

### Example 5

At least 10 Subjects who have previously taken 1 ml of a formulation having about 42 mg/ml of CBD (in the substantial absence of CBG and CBDV, e.g., <1 mg/ml) for insomnia or sleep disruption are given 1 ml of the formulation of Example 1. When polled the Subjects will likely report improvement in their sleep habits (e.g., more sleep) then compared to their sleep habits after being treated with the CBD formulation for about one month

### Example 6

At least five Subjects take 1 ml/day (preferably at night) of the formulation set forth in Example 1 for four days after experiencing muscle, joint and/or tendon soreness from exercise or trauma are expected to experience greater pain relief than when treated with 1 ml of a formulation having about 42 mg/ml of CBD in the substantial absence CBG and CBDV (less than 1 mg/ml of each).

### Example 7

At least five Subjects take 1 ml daily (preferably at night) of the formulation set forth in Example 2 for four days after experiencing muscle, joint or tendon soreness from exercise or trauma. Some of these patients are polled and are expected to experience longer and much greater pain relief than when treated with 1 ml of a formulation having about 42 mg/ml of CBD in the substantial absence of CBG and CBDV (less than 1 mg/ml each).

### Example 8

Four Subjects who experience indigestion, gas, Chron's Disease, IBS, diverticulitis, and/or GERD are given 1 ml of the formulation of Example 1 or 2 and are expected to obtain immediate relief (ten minutes or less) upon one dose of 1 ml of either formulation.

### Example 9

Sixteen subjects are giving at least 1 ml/day of the formulation of Example 4 for a period of 4 months, six subjects of which are given 2 ml/day. All subjects are expected to experience pain relief from muscle, joint or tendon soreness from exercise or trauma.

The expected results set forth in Examples 5-9 are based on preliminary polling of Subjects who have taken tinctures of Example 1, 2 or 4. The results appear to be unexpected and surprising as for example a formulation of Example 1 or 2 appears to be better than a formulation of CBD in the substantial absence of CBDV and CBG. Initial observations (verbal polling) may suggest synergistic results.

I claim:

1. An edible oil comprising:
1) from about 32 mg/ml to about 50 mg/ml of cannabigerol (CBG),
2) greater than about 32 mg/ml cannabidiol (CBD), and
3) from about 20 mg/ml to about 50 mg/ml of cannabidivarin (CBDV).

2. The composition as recited in claim 1 further comprising about 32 mg/ml to about 50 mg/ml THC-O-acetate.

3. The composition as recited in claim 1 wherein the oil comprises a medium chain triglyceride.

4. The composition as recited in claim 2 wherein the oil comprises a medium chain triglyceride.

5. The composition as recited in claim 1 wherein the oil comprises hemp oil.

6. The composition as recited in claim 2 wherein the oil comprises hemp oil.

7. An edible oil comprising:
1) about 39 mg/ml CBG,
2) about 95 mg/ml CBD,
3) About 22 mg/ml CBDV and
4) About 36 mg/ml THC-O-acetate.
